(19) United States
(12) Patent Application Publication
(10) Pub. No.: US 2024/0373858 A1
(43) Pub. Date: Nov. 14, 2024

(54) MICROBIAL INOCULANTS FOR PLANT GROWTH AND PATHOGEN RESISTANCE

(71) Applicant: FULL CIRCLE MICROBES, INC., HINESBURG, VT (US)

(72) Inventors: Victoria I. HOLDEN, Essex, VT (US); Charles SMITH, Winooski, VT (US); Katelynn KOSKIE, Essex Junction, VT (US); Rogelio ZIMBRON, Burlington, VT (US); Jack EVANS, Burlington, VT (US); Amy ZIOBRON, Burlington, VT (US)

(21) Appl. No.: 18/559,245

(22) PCT Filed: May 9, 2022
(86) PCT No.: PCT/US2022/028350

§ 371 Date: Nov. 6, 2023

Related U.S. Application Data
(60) Provisional application No. 63/185,677, filed on May 7, 2021.

(51) Int. Cl. A01N 63/22; A01N 25/12; A01N 63/25; A01N 63/27; A01N 63/30; A01N 63/38; A01P 21/00

(52) U.S. Cl. CPC A01N 63/22; A01N 25/12; A01N 63/25; A01N 63/27; A01N 63/30; A01N 63/38; A01P 21/00

(57) ABSTRACT

This disclosure describes microbial compositions that contain novel combinations of at least two microbial organisms that, when included in a microbial inoculate and applied to applied to seeds, plants, roots, or growth substrates, promotes plant growth or resistance to plant pathogens.

### CROSS REFERENCE TO RELATED APPLICATIONS

This application claims priority to U.S. Provisional Application No. 63/185,677, filed on May 7, 2021, the disclosure of which is incorporated by reference.

### TECHNICAL FIELD

The inventions described herein relate to microbial strains, compositions, and methods that are useful for promoting plant growth and/or plant pathogen resistance.

### BACKGROUND OF THE INVENTION

Worldwide, farmers spend over $376B on fertilizer and agricultural chemicals to stimulate crop yield and protect against pests (1). The application of these chemicals has significant unintended consequences for ecological health, amounting to $157B in damages to the environment and human health in the US alone (2). Pesticide exposure has been linked to higher rates of certain cancers (3) and 700% higher rate of miscarriages and birth defects (4). These harms most frequently impact communities of color, who are exposed to 56-63% more pollution than they produce (5).

Farmers face an economic and practical dilemma in their need to maintain high crop outputs, and they have used the tools available to them. The Agricultural industry requires new solutions to increase crop productivity without chemicals that damage the environment and human health. Optimized microbial communities offer a compelling alternative with the potential to benefit farmers as well as ecological health.

Soil health is a vital component to the lifetime performance of nearly all plants. In addition to providing water and a variety of nutrients essential to plant growth including nitrogen, potassium, and phosphorus, healthy soil also contains gases, organic matter, and microorganisms (6). Estimates from garden and farm soils show that one teaspoon of soil contains up to 1 billion bacteria and numerous yards of fungi (7).

Bacterial and fungal microorganisms living in the soil can act as decomposers participating in nutrient cycling, as mutualists forming beneficial relationships with plants, or as pathogens causing disease in the plants they infect (8,9). Bacterial mutualists are found throughout the soil, but are highly concentrated in the rhizosphere, the narrow region of soil next to and inside plant roots (10). Within the rhizosphere, roots release exudates to stimulate the growth of beneficial bacteria (10). Once established, these bacteria will promote plant growth by suppressing pathogens, converting nutrients like atmospheric nitrogen to bioavailable forms for plant uptake, and producing growth-promoting compounds like phytohormones (10).

The most common fungal mutualists are mycorrhizal fungi (10). Mycorrhizal fungi can live on the surface of roots (ectomycorrhizal) or inside roots (endomycorrhizal or arbuscular mycorrhizal) where they provide the root with access to water and nutrients in exchange for photosynthetically-derived carbohydrates (10). Mycorrhizal fungi can also suppress the growth of plant pathogens (11).

Certain mycorrhizal fungi and other beneficial microbes, including various bacterial species, may be useful for resolving problems associated with current fertilization practices that are facing the agricultural industry. The introduction of mycorrhizal fungi and other beneficial microbes can positively impact plants of industrial crops and home gardens alike. These plants include hops, ornamental flowers (roses, orchids, lavender, lilies, geranium, marigold), saffron, Cannabis, Christmas trees (fir trees), wine grapes, sunflowers, broccoli, rice, tomatoes, sugar cane, corn, wheat, soy, cotton, and tea (C. sinensis var. sinensis and C. s. var. assamica).

Cannabis represents a highly attractive use-case given the value of the crop, the increasing demand for Cannabis derivatives (including seeds, fiber, and cannabinoids), and the current opportunity to incorporate sustainable practices into the foundation of a rapidly expanding new segment of agriculture. It is, therefore, necessary to identify and develop such sustainable practices, including environmentally friendly alternatives to traditional fertilizers. Microorganisms, like mutualistic bacteria and mycorrhizal fungi, enhance biomass, nutrient acquisition, yield, and pathogen suppression in a variety of plants, and, thus, offer opportunities to reduce fertilizer usage in the agricultural industry (12-14).

### SUMMARY OF THE INVENTION

This disclosure describes embodiments of microbial compositions for promoting plant growth, or plant resistance to plant pathogens. In various embodiments, the microbial compositions contain a mixture of: at least one first microbial species selected from the group consisting of Azospirillum brasilense, Bacillus amyloliquefaciens, Pseudomonas fluorescens, Rhizophagus irregularis, Bradyrhizobium japonicum, Gluconacetobacter diazotrophicus, Pseudomonas putida, Rhodopseudomonas palustris, Trichoderma hamatum, Trichoderma virens, Bacillus subtilis, Rhizophagus diaphanus, Trichoderma reesei, and Laccaria bicolor; and at least one second microbial species selected from the group consisting of Azotobacter chroococcum, Herbaspirillum seropedicae, Paenibacillus lentimorbus, Sphingomonas paucimobilis, Pseudomonas chlororaphis, Azorhizobium caulinodans, Bacillus pumilus, Variovorax paradoxus, Rhizophagus intraradices, Rhizophagus clarus, and Trichoderma harzianum.

In some embodiments, a microbial composition of the invention promotes plant growth, and contains at least one first microbial species selected from one or more of Azospirillum brasilense, Pseudomonas fluorescens, Rhizophagus irregularis, Rhodopseudomonas palustris, Trichoderma hamatum, and Bacillus subtilis, and at least one second microbial species selected from one or more of Azotobacter chroococcum, Sphingomonas paucimobilis, Pseudomonas chlororaphis, Variovorax paradoxus, Rhizophagus intraradices, and Bacillus pumilus.

In other embodiments, a microbial composition of the invention promotes plant resistance to plant pathogens, in which:
a) the microbial composition contains at least one first microbial species selected from P. putida, B. subtilis, P. fluorescens and T. virens, and at least one second microbial species selected from B. pumilus and P. chlororaphis;
b) the microbial composition contains at least one first microbial species selected from P. putida, B. subtilis, P. fluorescens, T. virens, and, T. reesei, and at least one second microbial species selected from B. pumilus and P. chlororaphis; or
c) the microbial composition contains at least one first microbial species selected from A. brasilense, B. subtilis, and R. palustris, and at least one second microbial species selected from A. chroococcum, S. paucimobilis, and V. paradoxus;
d) the microbial composition contains at least one first microbial species selected from A. brasilense, P. fluorescens, B. subtilis, and R. palustris and at least one second microbial species selected from A. chroococcum, S. paucimobilis, P. chlororaphis, and V. paradoxus; or
e) the microbial composition contains at least one first microbial species selected from A. brasilense, P. fluorescens, and R. palustris and at least one second microbial species selected from A. chroococcum, S. paucimobilis, and P. chlororaphis.

In some embodiments of the invention, a microbial composition of the invention is lyophilized and resuspended in water or an aqueous solution to form a microbial inoculant, which may, optionally, include a carbon source and/or a thickening agent.

### BRIEF DESCRIPTION OF THE FIGURES

FIG. 1A depicts average whole plant fresh weight of hemp plants treated with either the Growth Enhancement or Pathogen Suppression inoculant in an outdoor field.

FIG. 1B depicts average bud dry weight of hemp plants treated with either the Growth Enhancement or Pathogen Suppression inoculant in an outdoor field.

FIG. 1C depicts average bud/fresh weight ratio of hemp plants treated with either the Growth Enhancement or Pathogen Suppression inoculant in an outdoor field.

FIG. 1D depicts average percent yield of hemp plants treated with either the Growth Enhancement or Pathogen Suppression inoculant in an outdoor field.

FIG. 2 depicts average Plant Growth Index of hemp treated with the Growth Enhancement inoculant in an outdoor field.

FIG. 3 depicts average cola counts of hemp plants treated with the Growth Enhancement and Pathogen Suppression inoculants in an outdoor field.

FIG. 4A depicts average blossom diameter of Zinnia plants treated with either the Growth Enhancement or Pathogen Suppression inoculant in an outdoor field.

FIG. 4B depicts average blossom diameter of Zinnia plants treated with either the Growth Enhancement or Pathogen Suppression inoculant in an outdoor field.

FIG. 5 depicts average blossom number of Zinnia plants treated with the Growth Enhancement inoculant.

FIG. 6 depicts average shoot length of field grown sunflower plants treated with the Growth Enhancement inoculant.

FIG. 7 depicts average plant growth index of field grown sunflower plants treated with the Growth Enhancement inoculant or both the rooting and growth enhancement inoculants.

FIG. 8 depicts results of plate competition assay for analyzing pathogen suppression of microbial inoculants.

FIG. 9 depicts average plant height of legume plants treated with growth enhancement inoculants.

FIG. 10 depicts average leaf surface area of legume plants treated with growth enhancement inoculants.

FIG. 11 depicts average biomass of legume plants treated with growth enhancement inoculants.

FIG. 12 depicts average biomass of hemp plants treated with growth enhancement inoculants.

FIG. 13 depicts average leaf surface area of hemp plants treated with growth enhancement inoculants.

FIG. 14 depicts average biomass of radish plants treated with growth enhancement inoculants.

FIG. 15 depicts average leaf surface area of radish plants treated with growth enhancement inoculants.

FIG. 16 depicts average leaf surface area of zinnia plants treated with growth enhancement inoculants.

FIG. 17 depicts average biomass of zinnia plants treated with growth enhancement inoculants.

FIG. 18 depicts average germination rate of corn plants treated with growth enhancement inoculants.

FIG. 19 depicts average germination rate of cosmos plants treated with growth enhancement inoculants.

FIG. 20 depicts average germination rate of legume plants treated with growth enhancement inoculants.

FIG. 21 depicts average germination rate of sunflower plants with growth enhancement inoculants.

FIG. 22 depicts results of plate competition assay for analyzing pathogen suppression of microbial inoculants.

### DETAILED DESCRIPTION

The inventions described herein relate to microbial compositions that contain combinations of microbial species, which when applied to seeds, plants, roots, or growth substrates (soils, hydroponic media, aeroponic media, etc.), promote certain desirable characteristics. A microbial composition of the invention may comprise, consist essentially of, or consist of a mixture of microbial species as described here. A microbial composition of the invention may have mixtures of microbial species that are particularly suited for promoting plant growth. While another microbial composition of the invention promotes resistance to organisms that are pathogenic to plants. In yet another microbial composition of the invention promotes plant growth and promotes resistance to plant pathogens. A microbial composition of microbial species may also be a lyophilized microbial composition of the microbial species.

Typically, microbial compositions of the invention promote plant growth by: (i) increasing the bioavailability of nutrients for uptake by the plants; (ii) altering the production or activity of plant hormones; or (iii) a combination of (i) and (ii). More generally, microbial compositions of the invention promote plant growth by beneficially interacting with the plants.

Plant growth promoted by a microbial composition of the invention may be quantitated by tracking one more growth outcomes, including, but not limited to an increase in plant biomass as measured by the quantification of plant height, width, and depth, and leaf size. Plant growth may also be evidenced by an increase in dry weight, as defined as the weight of the plant after harvest and after the remaining plant material has dried.

Typically, microbial compositions of the invention promote pathogen resistance by: (i) enhancing native defense and resistance systems of plants; (ii) introducing desirable microbes to outcompete undesirable microbes or pathogens; or (iii) a combination of (i) and (ii). Decreases in plant disease can be quantified by methods known in the art, including, but not limited to monitoring disease prevalence or severity across seasons, performing pathogen suppression assays, monitoring loss of plants or harvest yield due to disease caused by plant pathogens. More specifically, pathogen suppression assays may, but not necessarily, include a plate competition assay or a bioassay involving painting a small patch of pathogen on a plant leaf and monitoring subsequent disease progression, or a combination of such assays. And increases in yield, a term that is generally understood in the agricultural industry to mean sellable yield, may correspond to an increased harvest yield or increased plant biomass.

The terms “pathogen” or “plant pathogen” as used herein refers to an organism, such as an alga, an arachnid, a bacterium, a fungus, an insect, a nematode, a parasitic plant, a protozoan, a yeast, or a virus capable of producing a disease in a plant. In the agricultural arts, a plant pathogen is also commonly referred to as phytopathogen.

As disclosed above, a microbial composition of the invention contains a specified mixture of microbial species—typically fungal and bacterial species. For example, in certain microbial compositions of the invention, one or more species of a mycorrhizal fungi may be combined with: (i) another fungal species, including with one or more other species of mycorrhizal fungi; (ii) one or more bacterial species of bacteria; or (iii) a combination of fungal and bacterial species.

The microbial species of a microbial composition of the invention may have, but not necessarily, been isolated prior to being mixed into a composition with each other. More specifically, an isolated microbial species has been removed from its natural or culture milieu. Though, “isolated” does not necessarily reflect the extent to which the microbe has been purified.

In certain microbial compositions of the invention, the mixture of microbial species includes at least one microbial species—a first microbial species—selected from Azospirillum brasilense, Bacillus amyloliquefaciens, Pseudomonas fluorescens, Rhizophagus irregularis, Bradyrhizobium japonicum, Gluconacetobacter diazotrophicus, Pseudomonas putida, Rhodopseudomonas palustris, Trichoderma hamatum, Trichoderma virens, Bacillus subtilis, Rhizophagus diaphanus, Trichoderma reesei, and Laccaria bicolor; and at least one microbial species—a second microbial species—selected from Azotobacter chroococcum, Herbaspirillum seropedicae, Paenibacillus lentimorbus, Sphingomonas paucimobilis, Pseudomonas chlororaphis, Azorhizobium caulinodans, Bacillus pumilus, Variovorax paradoxus, Rhizophagus intraradices, Rhizophagus clarus, and Trichoderma harzianum.

For example, a particular microbial composition of the invention useful for enhancing plant growth and/or pathogen resistance may contain at least one first microbial species selected from Azospirillum brasilense, Pseudomonas fluorescens, Rhizophagus irregularis, Rhodopseudomonas palustris, Trichoderma hamatum, and Bacillus subtilis that is combined with at least one second microbial species selected from Azotobacter chroococcum, Sphingomonas paucimobilis, Pseudomonas chlororaphis, Variovorax paradoxus, Rhizophagus intraradices, and Bacillus pumilus.

Indeed, in a preferred microbial composition of the invention for enhancing plant growth, the composition contains (i.e., comprises) A. brasilense, B. subtilis, and R. palustris, and A. chroococcum, S. paucimobilis, and V. paradoxus. Another preferred microbial composition according to the invention for promoting plant growth contains (i.e., comprises) A. brasilense, P. fluorescens, B. subtilis, and R. palustris and A. chroococcum, S. paucimobilis, P. chlororaphis, and V. paradoxus. Another preferred microbial composition according to the invention for promoting plant growth contains (i.e., comprises) A. brasilense, P. fluorescens, and R. palustris and A. chroococcum, S. paucimobilis, and P. chlororaphis. Similarly, a microbial composition of the invention may consist of, or consist essentially of (A. brasilense, B. subtilis, and R. palustris, and A. chroococcum, S. paucimobilis, and V. paradoxus), (A. brasilense, P. fluorescens, B. subtilis, and R. palustris and A. chroococcum, S. paucimobilis, P. chlororaphis, and V. paradoxus), or (A. brasilense, P. fluorescens, and R. palustris and A. chroococcum, S. paucimobilis, and P. chlororaphis).

Alternatively, in a preferred microbial composition of the invention for promoting pathogen resistance, the composition contains (i.e., comprises) P. putida, B. subtilis, P. fluorescens and T. virens, and B. pumilus and P. chlororaphis. Another preferred microbial composition of the invention for promoting plant pathogen resistance is P. putida, B. subtilis, P. fluorescens, T. virens, and, T. reesei, and B. pumilus and P. chlororaphis. Similarly, a microbial composition of the invention for promoting plant pathogen resistance may consist of, or consist essentially of (P. putida, B. subtilis, P. fluorescens and T. virens, and B. pumilus and P. chlororaphis) or (P. putida, B. subtilis, P. fluorescens, T. virens, and, T. reesei, and B. pumilus and P. chlororaphis).

The microbial species used in a microbial composition or a microbial inoculant of the invention themselves can originate from a frozen glycerol stock, a solid-medium growth plate, or a commercially available source. A microbial species can also be isolated from environmental samples or purchased from open-access or other available culture collections. The selected microbial species can then be streak-plated in a sterile environment on a petri dish or other containers of solid media to generate single colony isolates. Streak-plated samples on petri dishes or other containers can be incubated and isolated using techniques known in the art. For example, a microbial species may be incubated for 24-48 hours or longer at 30° C. aerobically, at 37° C. under normal atmospheric conditions, or at any other condition optimal or sufficient for colony formation for a given species or strain. After incubation and colony formation, individual colonies can be isolated for propagation in liquid media for a further 24-48 hours or longer as stated above. Isolated microbial species or strains can be stored at −80° C. in 25-50% glycerol for continued propagation. Media and growth conditions can vary and are preferably optimized for a given strain. Media can be used for culturing, isolating, and storing microbes. Suitable media can be comprised of a carbon source, an amino acid source, salts, buffers, and yeast or meat extracts. Media can be prepared as a liquid or as a solid by supplementing with agar.

The microbial species used in a microbial composition or a microbial inoculant of the invention can be cultured in media comprising exogenous tryptophan. Some microbial species used in a microbial composition or a microbial inoculant of the invention are capable of enzymatically converting tryptophan to indoleacetic acid (IAA)—a plant hormone capable of increasing rooting capacity and promoting root growth. Accordingly, the presence of exogenous tryptophan in growth media can lead to increased production of IAA, thereby increasing capacity and promoting root growth in plants to which the mixture of microbial species or microbial inoculant are applied.

In a microbial composition or a microbial inoculant of the invention, individual strains or species can be present in equal concentrations. Alternatively, individual strains or species can be present in >1 to 1,000-fold excess over another strain or species present, in >1 to 500-fold excess, in >1 to 100-fold excess, in >1- to 50-fold excess or in >1- to 10-fold excess.

As discussed above a microbial composition containing a mixture of microbial species according to the invention may be a mixture of individually lyophilized microbial species. As known in the art lyophilization is a process by which water is removed by freezing the material and then reducing the pressure and adding heat to allow the frozen water in the material to sublimate. Lyophilization can be used to preserve perishable material, including microbes, and make it more convenient for transport. Preparation of the lyophilized mixture can be accomplished by inoculating, growing, pelleting, and lyophilizing individual species or strains before combining the lyophilized materials to form the lyophilized mixture. Strains of the same species can be combined after pelleting and before lyophilization, or after pelleting and lyophilization.

Starter cultures for lyophilization mixture can be prepared by inoculating a strain from a frozen glycerol stock or solid growth plate into liquid media for example using 5-500 mL volume or other volumes known in the art. Likewise, starter culture volume can be for example <5 mL or >500 mL or other volumes known in the art. Starter cultures can be used to inoculate a bulk culture that is for example 20-50 L in volume or other volumes known in the art. Likewise bulk culture can be for example <20 L or >50 L or other volumes known in the art. Bulk culture can be cycled through multiple draw/fill cycles as desired. Draw/fill cycles involve growing the culture to the desired cell density, removing a portion of the culture, and supplementing the remainder with fresh media for continued growth. Once desired cell density is reached, microbes can be pelleted from media by centrifugation. Strains of the same species can be optionally combined, and pellets can be resuspended in, for example, 2 L of media and lyophilized. Resuspension volume can be resuspended in for example volumes <2 L or >2 L or volumes depending on the capacity of lyophilization equipment. Individual lyophilized microbial species can be combined to generate the final lyophilized mixture. Lyophilized mixture can be packaged in packets for subsequent distribution and use.

The invention also relates to microbial inoculants. As understood herein, a microbial inoculant contains, at minimum, suspension of a microbial composition of the invention in water or an aqueous solution. Thus, an inoculant of the invention may be, for example, prepared by resuspending a microbial composition in water. Resuspension volume can be for example <1 gallon (U.S. Customary Units equivalent to 3.785 L) or >1 gallon or other volume, such as, but not limited to 200 ml, 250 ml, 300 ml, 350 ml, 400 ml, 450 ml, 500 ml, 550 ml, 600 ml, 650 ml, 700 ml, 750 ml, 800 ml, 850 ml, 1000 ml, 1250 ml, 1500 ml, 1750 ml, 2000 ml, 2250 ml, 2500 ml, 2750 ml, 3000 ml, 3250 ml, 3500 ml, 3750 ml, 4000 ml, or any volume therein.

Optionally, a microbial inoculant of the invention may also contain a carbon source. In other words, a microbial inoculant of the invention may be supplemented with one or more carbon sources. The carbon source of an inoculant of the invention may be admixed with a lyophilized microbial composition or it may be added at the time a lyophilized microbial composition is resuspended in water or an aqueous solution. Examples of carbon sources include, but are not limited to, hexoses, such as glucose, but other sources that are readily assimilated, such as amino acids, may also serve as a carbon source. The amount of a carbon source in a microbial inoculant of the invention may vary depending on the particular combination of microbial species in a microbial composition of the invention; accordingly, the invention does not specify a limit on the total amount of a carbon in a microbial inoculant of the invention. However, in one microbial inoculant of the invention, a carbon source, when present, may constitute 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of microbial inoculant (weight/volume). Accordingly, the amount of a carbon source in some microbial inoculants of the invention be from 9-15%, 10-14%, or 11-13% (w/v).

Optionally, a microbial inoculant of the invention may also contain a thickening agent, such as, but not limited to aloe vera, aloe vera flakes, willow bark extract, silica, pectin, psyllium husk, or a gelling agent. A thickened microbial inoculant can be more easily applied to a cutting or roots thereof. The amount of a thickening agent in a microbial inoculant of the invention may vary depending on the particular use of the inoculant; accordingly, the invention does not specify a limit on the total amount of a thickening agent in a microbial inoculant of the invention. However, in one microbial inoculant of the invention, a thickening agent, when present, may constitute 1%, 2%, 3%, 4%, 5%, 6%, 7%, 8%, 9%, 10%, 11%, 12%, 13%, 14%, 15%, 16%, 17%, 18%, 19%, or 20% of microbial inoculant (weight/volume). Accordingly, the amount of a carbon source in some microbial inoculants of the invention be from 2-8%, 3-7%, or 4-6% (w/v).

Once prepared, microbial inoculants can be poured, sprayed, or otherwise applied to ungerminated seeds, germinated seeds, seedlings, cuttings, roots, maturing or young plants, maturing plants at a time before or at the time of flowering, or a growth substrate in either a greenhouse, field, hydroponic grow space, indoor grow space, aeroponic grow space, or other grow space. Microbial inoculants can be applied to seeds or roots before planting by bathing the seeds or roots in the liquid form. Microbial inoculants can be applied to the germinated seed upon planting by inoculating the growth substrate with the liquid form. The growth substrate can be a mixture comprising at least one of soil, perlite, coco coir, vermiculite, pumice, peat moss, compost, aqueous growth media, rock wool, and worm casting or other growth substrates known in the art. Microbial inoculants in liquid or spray form can be applied to the seedling, cutting, or roots before or at the time of transplanting or propagation. Transplanting can be uprooting and transferring the seedling from one growth substrate or grow space to another. Microbial inoculants can be applied to young or maturing plants as a foliar spray or as a liquid applied to the growth substrate or directly to the roots and/or aerial (e.g. above soil) plant tissue. Microbial inoculants can be applied to the mature plant at time of flowering as a foliar spray or as a liquid applied to the growth substrate. Microbial inoculants can be applied to mature plants prior to harvest as a foliar spray or as a liquid applied to the growth substrate. Microbial inoculants can be applied to the soil post-harvest in liquid or spray form to inoculate or “prime” the soil or other growth substrate for the next growing season. Priming the soil or other growth substrate allows for microbial communities to be established prior to planting.

Microbial inoculants can be applied as described repeatedly. Application of the microbial inoculants can occur seasonally, bimonthly, monthly, every 3 weeks, every 2 weeks, weekly, every 5 days, every 3 days, daily, or a similar such period of time appropriate for the grow space and plant species.

In certain instances, the concentration of microbes in a repeatedly applied inoculant can differ from that of the originally applied inoculant. Concentration of microbes in a repeated inoculant can differ relative to that of a previously applied inoculant by a factor of >1 to 1,000, by a factor of >1 to 500, by a factor of >1 to 100, by a factor of >1 to 50, by a factor of >1 to 10, or by a factor <1.

### EXAMPLES

### Example 1: Lyophilized Microbial Mixture Production—General Protocol

Bacterial and/or fungal strains are inoculated from frozen glycerol stock or solid growth plate into 5-500 mL liquid media appropriate for each strain. Strains are inoculated from 5-500 mL liquid culture into 20-50 L liquid culture. Cultures are cycled through draw/fill growth cycles as desired. Strains are pelleted by centrifugation and combined into a single 2 L media suspension. The combined 2 L suspension is lyophilized. These steps are repeated for each individual microbial species that is to comprise the inoculant. All lyophilized species are combined to generate a final lyophilized microbial composition. Final concentrations of inoculant species are equal compared to one another and at least OD₆₀₀=0.7 (0.7×10⁸ CFU/mL). Lyophilized inoculant is packaged as packets for subsequent use.

Media for each microbe can be comprised of the components as described in Table 1, or can differ according to best practice known in the art.

TABLE 1
Media Compositions

| Microbes | Media Name | Media Components |
|---|---|---|
| Bacillus subtilis | Nutrient | Beef extract |
| Rhodopseudomonas palustris | | Peptone |
| Pseudomonas putida | | |
| Sphingomonas paucimobilis | | |
| Variovorax paradoxus | | |
| Pseudomonas chlororaphis | | |
| Pseudomonas fluorescens | | |
| Bacillus pumilus | | |
| Azorhizobium caulinodans | | |
| Azospirillum brasilense | | |
| Bacillus amyloliquefaciens | | |
| Azotobacter chroococcum | Nitrogen- | K₂HPO₄ |
| | free | MgSO₄•7H₂0 |
| | | CaCO₃ |
| | | NaCl |
| | | FeSO₄•7H₂0 |
| | | NaMO₄•2H₂0 |
| | | Glucose |
| Herbaspirillum seropedicae | Spirillum | KH₂PO₄ |
| | nitrogen- | K₂HPO₄ |
| | fixing | MgSO4•7H₂0 |
| | medium | NaCl |
| | | CaCl₂ |
| | | FeCl₃ |
| | | Na₂MoO₄•2H₂0 |
| | | Sodium malate |
| | | Yeast extract |
| Paenibacillus lentimorbus | Bacillus | Mueller-Hinton broth |
| | Lentimorbus | Yeast extract |
| | Media | K₂HPO₄ |
| | | Glucose |
| | | Sodium pyruvate |
| Bradyrhizobium japonicum | Rhizobium | Yeast extract |
| | Medium | Mannitol |
| | | Air-dried garden soil |
| | | Na₂CO₃ |
| Gluconacetobacter | Sabouraud | SABOURAUD-2% |
| diazotrophicus | Glucose | Glucose-Bouillon |
| Rhizophagus diaphanus | Soil/Sand | Root tissue |
| Rhizophagus intraradices | Culture | Soil |
| Rhizophagus clarus | | Sand |
| Rhizophagus irregularis | | |
| Trichoderma hamatum | | |
| Trichoderma virens | | |
| Trichoderma harzianum | | |
| Laccaria bicolor | | |
| Rhizophagus diaphanus | MSR | MgSO₄•7H₂0 |
| Rhizophagus intraradices | | KNO₃ |
| Rhizophagus clarus | | KCl |
| Rhizophagus irregularis | | KH₂PO₄ |
| | | Ca(NO₃)₂•4H₂0 |
| | | Panthotenate Ca |
| | | Biotine |
| | | Nicotinic acid |
| | | Pyridoxine |
| | | Thiamine |
| | | Cyanocobalamine |
| | | Na Fe EDTA |
| | | MnSO₄•4H₂0 |
| | | ZnSO₄•7H₂0 |
| | | H₃BO₃ |
| | | CuSO₄•5H₂0 |
| | | Na₂MoO4•2H₂0 |
| | | (NH4)₆Mo₇O₂₄•4H₂0 |
| | | Sucrose |
| Trichoderma hamatum | Potato | Potato infusion |
| Trichoderma virens | Dextrose | Glucose |
| Trichoderma harzianum | | |
| Laccaria bicolor | Malt Media, | malt extract, or |
| | or MS | Murashige and |
| | Media | Skoog powder |

### Example 2: Preparation of a Microbial Inoculant

Lyophilized packet is resuspended in 1 gallon of water or a volume depending upon the area to be inoculated. Optionally, this suspension is supplemented with a sugar and/or carbon source. This forms the “microbial inoculant” for direct application methods of the invention.

### Example 3: Direct Application Methods

### 3.1 Application to Ungerminated Seed or Germinated Seed Upon Planting

The microbial inoculant is applied directly to ungerminated or germinated seed planted directly in soil or other growth substrate (either potted or grounded).

### 3.2 Application to Seedling at Transplanting

The microbial inoculant is applied directly to seedlings transplanted into soil or other growth substrate (either potted or grounded).

### 3.3 Application to Cutting

The microbial inoculant is applied directly to the cutting at the cut sites, or into the substrate or container into which the cutting is transplanted.

3.4 Application Directly to Maturing or Young Plants after Planting and/or Before Flowering

The microbial inoculant is sprayed or otherwise applied directly onto the growth substrate in either a greenhouse, field, hydroponic grow space, indoor grow space, aeroponic grow space, or other grow space.

### 3.5 Application Directly to Growth Substrate Post-Harvest for Priming Purposes

Inoculant is sprayed or otherwise applied directly to the growth substrate in a greenhouse, field, hydroponic grow space, indoor grow space, aeroponic grow space, or other grow space after crops have been harvested.

### Example 4: Treatment of Hemp Plants with Microbial Inoculants

### 4.1 Preparation and Application of Microbial Inoculants

Microbial inoculants were prepared as follows. Microbes comprising each inoculant were grown individually in liquid culture and combined at an equal ratio based on OD600 nm to create a final liquid culture with an OD600 nm=1.

The Growth Inoculant was comprised of the following microbial species: A. brasilense, A. chroococcum, B. subtilis, R. palustris, S. paucimobilis, and V. paradoxus.

The Pathogen Suppression inoculant was comprised of the following microbial species: P. putida, B. pumilus, P. chlororaphis, B. subtilis, P. fluorescens, T. virens, and T. reesei.

This inoculant was then applied with garden sprayers mostly as a foliar spray with some sprayed at the crown to inoculate the soil below. The Growth Enhancement Inoculant was applied 3 times approximately every 4 weeks between late May and early August, and the Pathogen Suppression Inoculant was applied 9 times approximately every 2 weeks between mid-May and mid-September.

### 4.2 Results

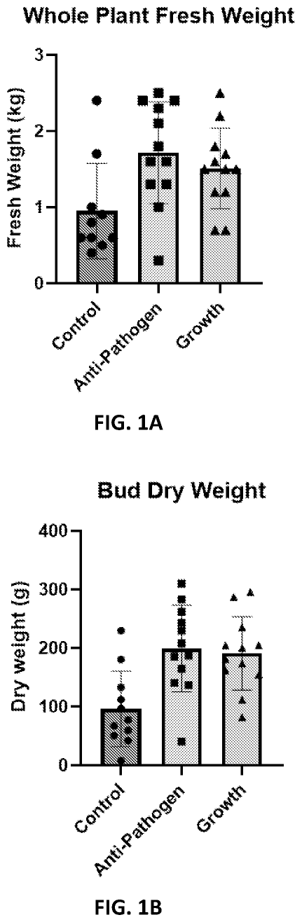

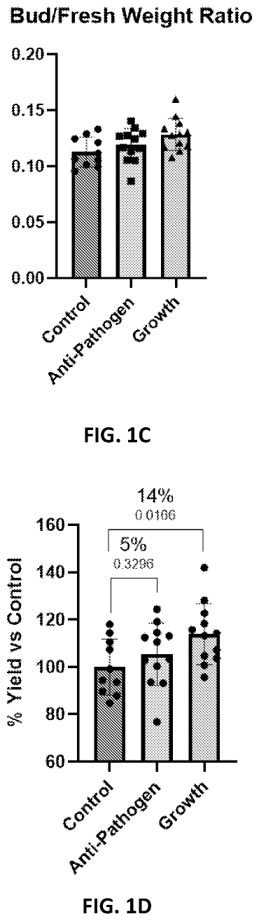

Hemp treated with either the Growth Enhancement or Pathogen Suppression inoculant in an outdoor field resulted in increases in both whole plant fresh weight (FIG. 1A) and dried bud weight (FIG. 1B) when compared to the control group. The control was treated with microbial growth media only. In addition, treatment with both inoculants resulted in more bud-dense plants (FIG. 1C). For FIGS. 1A-1D, each solid bar represents the average whole plant fresh weight, bud dry weight, bud/fresh weight ratio, and the percent yield increase with individual values (n≥10). Bud density was measured as dried bud/fresh weight ratio.

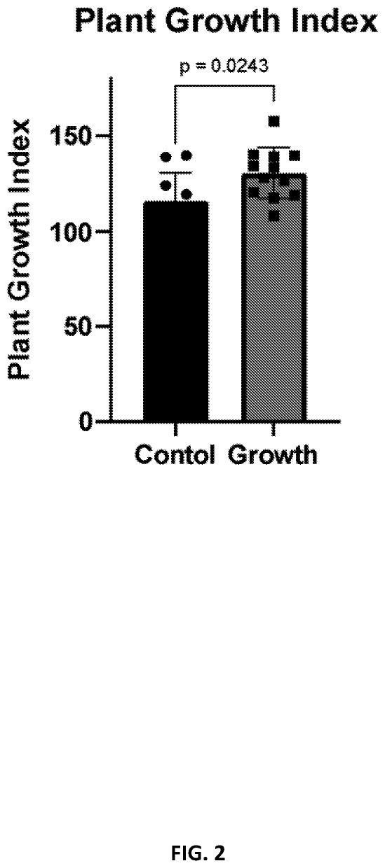

Hemp treated with the Growth Enhancement inoculant in an outdoor field results in an increase in Plant Growth Index (PGI) when compared to the control (FIG. 2). Each solid bar represents the average PGI with individual values (n≥10). Plant Growth Index is calculated by averaging together each plant's height, width, and depth.

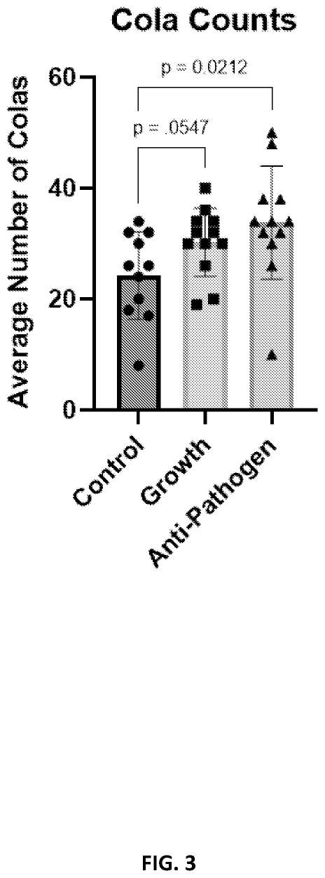

Hemp treated with the Growth Enhancement and Pathogen Suppression inoculants in an outdoor field resulted in an increase in the average number of colas per plant when compared to the controls (FIG. 3). Each solid bar represents the average number of colas per plant with individual values (n 11).

Hemp treated with the Pathogen Suppression inoculant in an outdoor field provided early protection from Septoria compared to the control (Table 2). Leaf incidence was determined by presence or absence of Septoria. Average severity was calculated subjectively on a scale of 1 to 100 with 1 being the least.

TABLE 2
Average Infection by Septoria in Hemp

| Group | Average Leaf Incidence | Severity (1 to 100) |
|---|---|---|
| Control | 0.2666666667 | 7.166666667 |
| Anti-Pathogen | 0 | 0 |

Hemp treated with the Pathogen Suppression inoculant in an outdoor field resulted in early suppression of Septoria as measured by the average number of leaves affected when compared to the control (Table 3). The average whole plant severity score was calculated in a scale of 1 to 5 with 1 being less infected by Septoria or bud rot. Bud rot data showed comparable results between the Pathogen Suppression and control groups.

TABLE 3
Average Hemp Infection by Septoria and Bud Rot (8.20.2021)

| | Average | Average | |
|---|---|---|---|
| | Number of | Whole Plant | Average |
| | Leaves | Severity Score | # of Colas |
| Group | Affected | (1 to 5) | Infected |
| Control | 20.45454545 | 2.090909091 | 0.09090909091 |
| Anti-Pathogen | 13.33333333 | 1.916666667 | 0.08333333333 |

### Example 5: Treatment of Blueberries and Strawberries with Pathogen Suppression Inoculant

Microbial inoculant preparation and application was as described in Example 4. Blueberries treated once with the Pathogen Suppression inoculant in an outdoor field resulted in a reduction in stem canker symptoms compared to the control (Table 4). The stem incidence was calculated by counting the number of infected branches, and the stem severity score was calculated in a scale of 1 to 100 with 1 being the least.

TABLE 4
Average Stem Infection by Canker in Blueberries

| Group | Stem Incidence | Severity (1 to 100) |
|---|---|---|
| Control | 6.4 | 1 |
| Anti-Pathogen | 4.4 | 0.8 |

Strawberries treated with the Pathogen Suppression inoculant in an outdoor field had less severe common leaf spot symptoms compared to the control (Table 5). Average stem incidence represents the number of common spot infection sites in the stem. Average severity was calculated subjectively on a scale of 1 to 100 with 1 being the least. No fruits were present on the plants.

TABLE 5
Average Infection by Common Spot in Strawberries

| | | | | | Fruit | |
|---|---|---|---|---|---|---|
| | Average # | Severity | Stem | | Inci- | |
| | of Leaf | (1 to | Inci- | | dence | |
| Group | Spots | 100) | dence | Severity | (no fruits) | Severity |
| Control | 24.2 | 3.8 | 1 | 1.2 | 0 | 0 |
| Anti- | 24.6 | 2.6 | 0.4 | 0.4 | 0 | 0 |
| Pathogen | | | | | | |

### Example 5: Treatment of Zinnia Plants with Growth Enhancement and Pathogen Suppression Inoculants

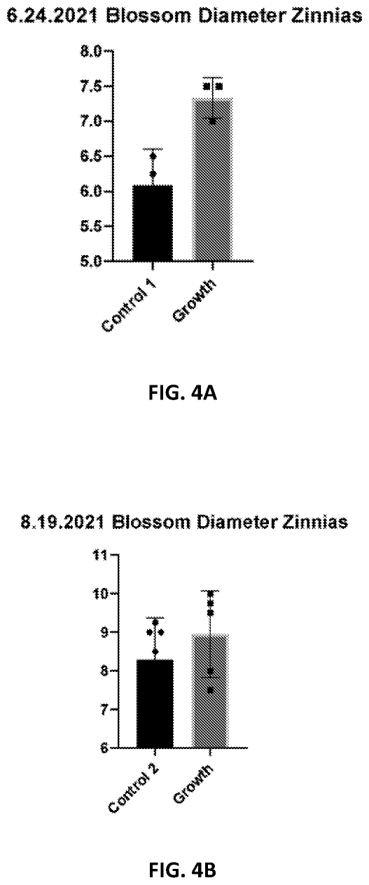

Microbial inoculant preparation and application were as described in Example 4. Zinnias treated with the Growth Enhancement Inoculant displayed an increase in average blossom diameter compared to the control (FIG. 4A-4B. Each solid bar represents the average blossom diameter with individual values (n≥3).

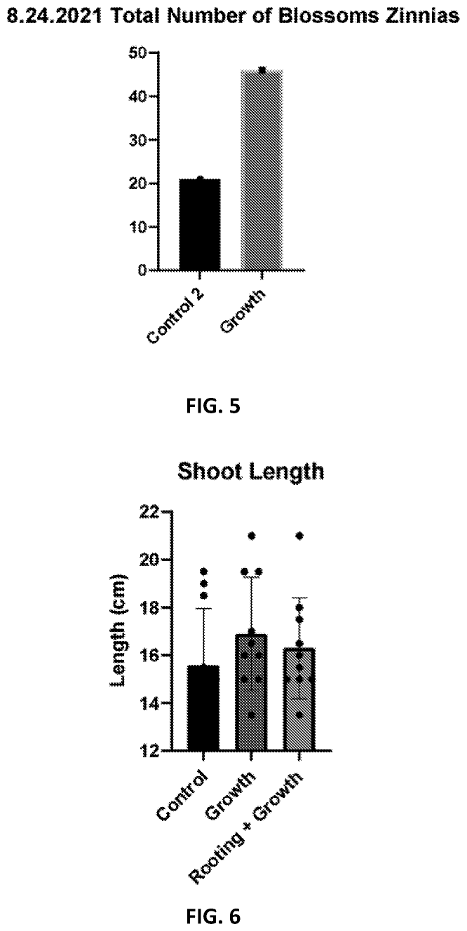

Zinnias treated with the Growth Enhancement Inoculant also had an increase number of blossoms (FIG. 5). Each solid bar represents the total number of blossoms. Zinnias were continuous harvested.

Zinnias treated with the Full Circle Microbes Pathogen Suppression Inoculant resulted in a decrease in leaf infection severity and comparable stem infection severity by Sclerotinia when compared to control plants (Table 6A). Additionally, powdery mildew was partially suppressed as measured by average leaf severity in plants treated with the Pathogen Suppression Inoculant when compared to controls (Table 6B). Average leaf and stem incidence were measured by whether or not a leaf or stem was infected, and average leaf and stem severity was measured on a scale of 1 to 100. Plants infected with Sclerotinia had also been pruned prior to this collection time point.

### Table 6A and 6B:

A. Leaf and Stem Infection by Sclerotinia

| | | Average | | Average |
|---|---|---|---|---|
| | | Leaf | | Stem |
| | Average | Severity | Average | Severity |
| | Leaf | Score | Stem | Score |
| Group | Incidence | (1 to 100) | Incidence | (1 to 100) |
| Control | 0.1703703704 | 14.81481481 | 0.2 | 20 |
| Anti- | 0.1 | 10 | 0.2 | 20 |
| Pathogen | | | | |

B. Leaf Infection by Powdery Mildew

| | Average | Average Leaf Severity |
|---|---|---|
| Group | Leaf Incidence | Score (1 to 100) |
| Control | 0.6333333333 | 2.866666667 |
| Anti- | 0.6666666667 | 1.633333333 |
| Pathogen | | |

### Example 6: Treatment of Redeemer Wheat with Pathogen Suppression Inoculant

Microbial inoculant preparation and application were as described in Example 4. Redeemer wheat treated with the Pathogen Suppression Inoculant showed reduced infection severity by common smut compared to the control group (Table 7). Average severity was scored from 1 to 100 with 1 being the least severe.

TABLE 7
Redeemer Wheat Infection

| | Whole | | Whole | |
|---|---|---|---|---|
| | Plant | | Plant | |
| | Incidence | | Incidence | Average |
| | (white | Average | (common | Severity |
| Group | fungus) | Severity | smut) | (1 to 100) |
| Control | 0.2 | 0.2 | 1 | 11.6 |
| Anti- | 0.06666666667 | 0.06666666667 | 1 | 6.133333333 |
| Pathogen | | | | |

### Example 6: Treatment of Field Grown Sunflowers with Growth Enhancement Inoculant

Microbial inoculant preparation and application were as described in Example 4. Field grown sunflowers treated with the Growth Enhancement Inoculant showed an increase in shoot length compared to the control (FIG. 6). Each solid bar represents the average shoot length with individual values (n≥10). Combining the Growth Enhancement and Root Enhancing inoculants also resulted in an increase in shoot length compared to the control. Combined Growth and Root Enhancement inoculants were comprised of the following microbial species: A. brasilense, P. lentimorbus, P. putida, T. virens, A. chroococcum, B. subtilis, R. palustris, S. paucimobilis, and V. paradoxus.

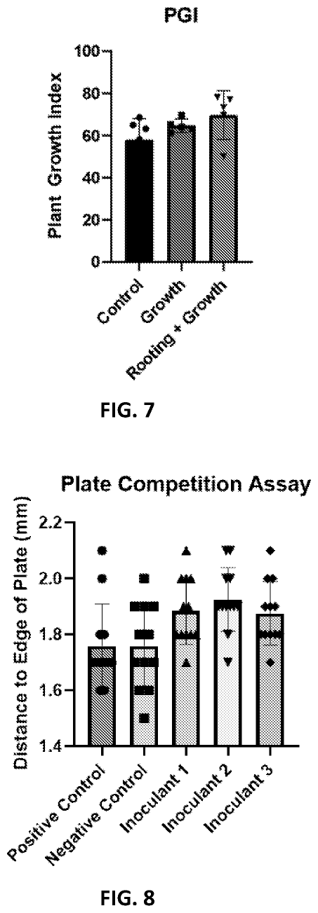

Field grown sunflowers treated with the Growth Enhancement Inoculant showed an increase in Plant Growth Index compared to the control (FIG. 7). Each solid bar represents the Plant Growth Index with individual values (n 5). Combining the Growth Enhancement and Root Enhancing inoculants also resulted in an increase in PGI compared to the control.

### Example 7: Plate Competition Assays

7.1

Three versions of a pathogen suppression inoculant were prepared as described in Example 4. All three versions of the Pathogen Suppression Inoculant inhibited Rhizoctonia solani in microbe-microbe plate competition assays compared to the negative controls (FIG. 8). Each solid bar represents the average distance from the edge of the plate to the border of R. solani growth with individual values (n 12). Four dots of inoculant were place around the edge of the plate, then a plug of R. solani was added the following day. When the R. solani reached halfway to the edge of the plate in the negative controls (about 2 days at room temperature) the data was collected. The greater the distance from the edge of the plate to the R. solani, the great the inhibition. All Pathogen Suppression inoculants inhibited R. solani compared to the negative control. Inoculants and controls were comprised of the following microbial species. Positive control: P. chlororaphis; Negative control: No microbes; Inoculant 1: P. putida, B. pumilus, P. chlororaphis, B. subtilis, P. fluorescens; Inoculant 2: B. pumilus, P. chlororaphis, P. lentimorbus; Inoculant 3: P. putida, B. subtilis, P. fluorescens, P. lentimorbus.

7.2

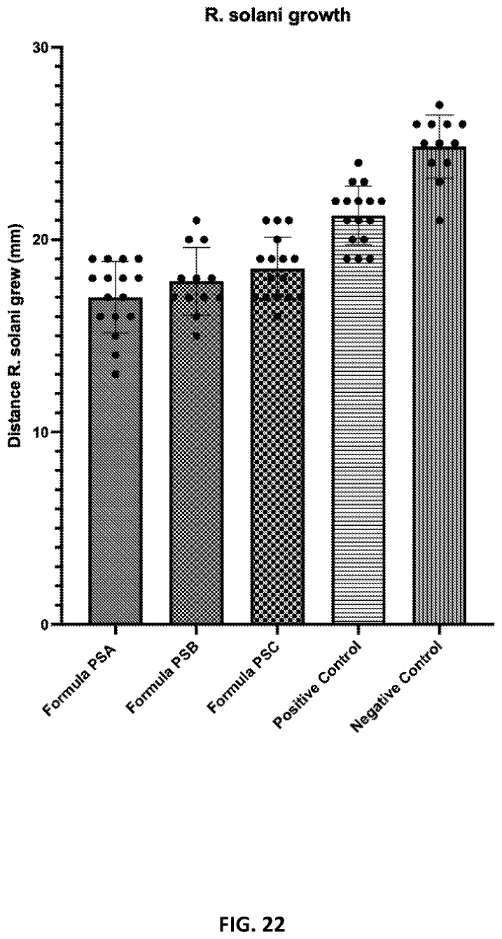

Three additional versions of a pathogen suppression inoculant were prepared as described in Example 4. All three formulations of the Pathogen Suppression inoculant were more effective at inhibiting R. solani growth compared to the controls (FIG. 22). Each solid bar represents the average distance from the center of the agar plate to the edge of the R. solani growth. Four, 20 μL droplets of the pathogen suppression inoculants were placed on the agar, and R. solani was added 2 days later. Measurements were taken 2 days later when R. solani reached halfway to the edge of the negative control plates. The smaller the distance from the center of the plate to the edge of R. solani growth indicates greater inhibition. Methods were modified from Neher et al. (15). Inoculants and controls were comprised of the following microbial species. Formula PSA: P. fluorescens, P. putida, P. chlororaphis, B. pumilus, B. subtilis; Formula PSB: P. fluorescens, P. putida, B. pumilus, B. subtilis; Formula PSC: P. fluorescens, P. chlororaphis, B. pumilus; Positive Control: P. chlororaphis; Negative Control: No bacteria applied.

### Example 8: Treatment of Legume Plants with Growth Enhancement Inoculants

Growth enhancement inoculants were prepared and applied as described in Example 4. Formula GRA was comprised of the following microbial species: A. brasilense, P fluorescens, R. palustris, B. subtilis, A. chroococcum, S. paucimobilis, P. chlororaphis, V. paradoxus. Formula GRB was comprised of the following microbial species: A. brasilense, P fluorescens, R. palustris, A. chroococcum, S. paucimobilis, P. chlororaphis.

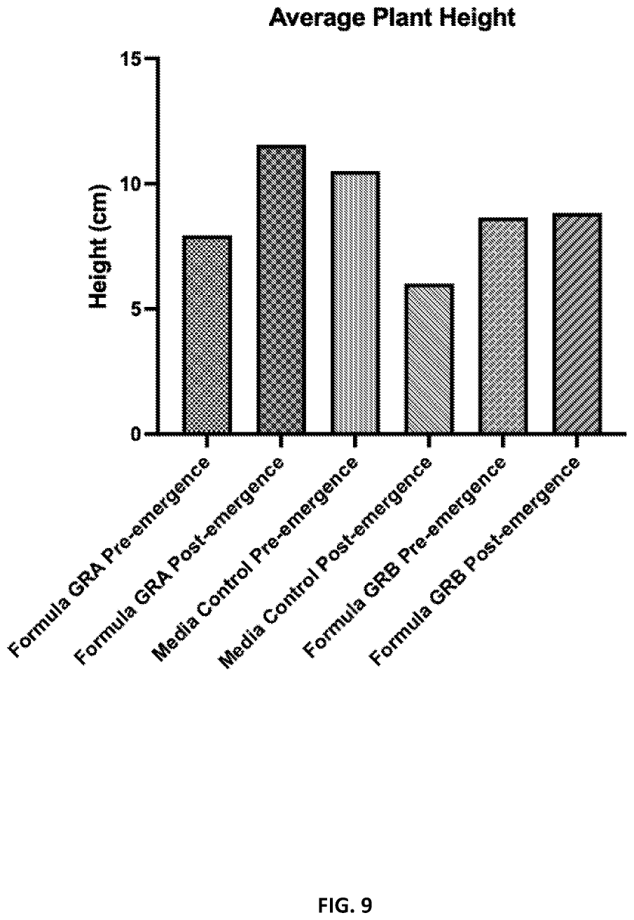

Formula GRA applied post germination was the most effective at enhancing the height of the legumes (FIG. 9). Each bar represents the average height of the legumes. Seeds were planted and 10 mL of microbial inoculants or controls were applied to the soil either just after planting or after they had emerged from the soil. Measurements were taken 10 days post germination, from the base of the plant.

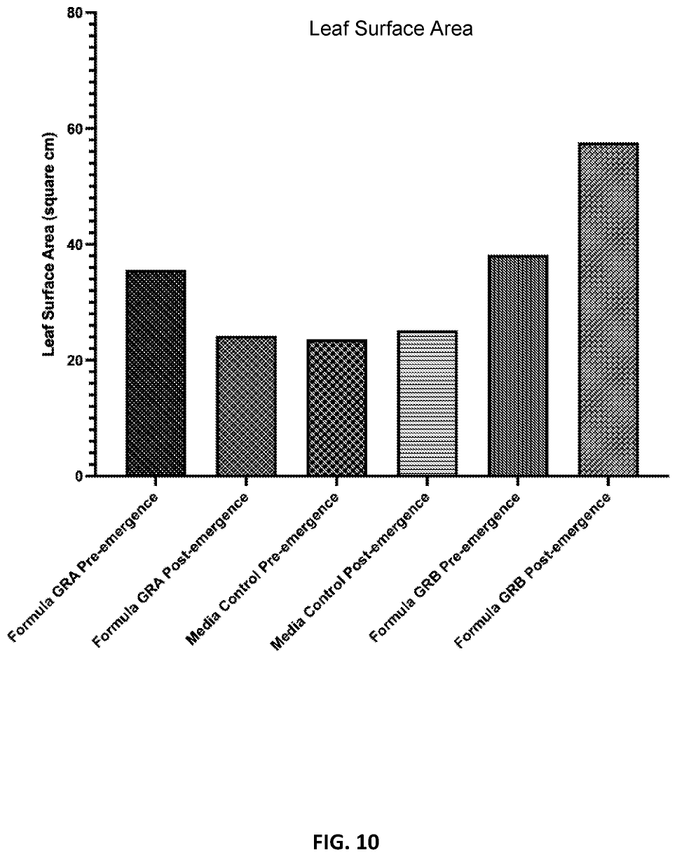

Formula GRB applied post germination was the most effective leaf surface area enhancer (FIG. 10). Each bar represents the average leaf surface area of the legumes. Seeds were planted and 10 mL of microbial inoculants or controls were applied to the soil either just after planting or after they had emerged from the soil. Measurements were taken 10 days post germination using the imaging and analytical software ImageJ.

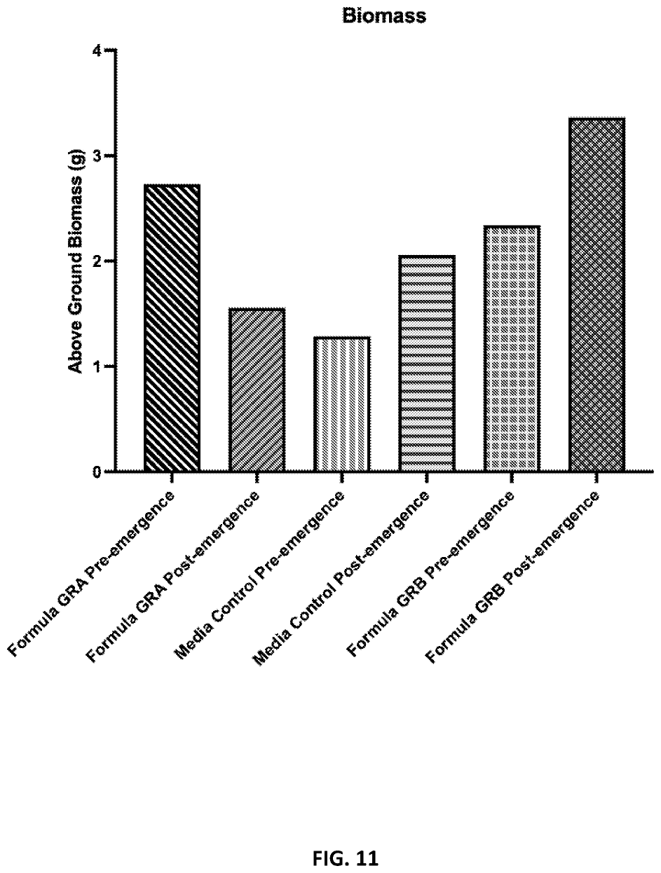

Formula GRB applied post germination was the most effective biomass enhancer (FIG. 11). Each bar represents the average biomass of the legumes. Seeds were planted and 10 mL of microbial inoculants or controls were applied to the soil either just after planting or after they had emerged from the soil. Biomass was measured by cutting all of the seedlings and weighing them on an analytical scale. Measurements were taken 10 days post germination, from the base of the plant

### Example 9: Treatment of Hemp Plants with Growth Enhancement Inoculants

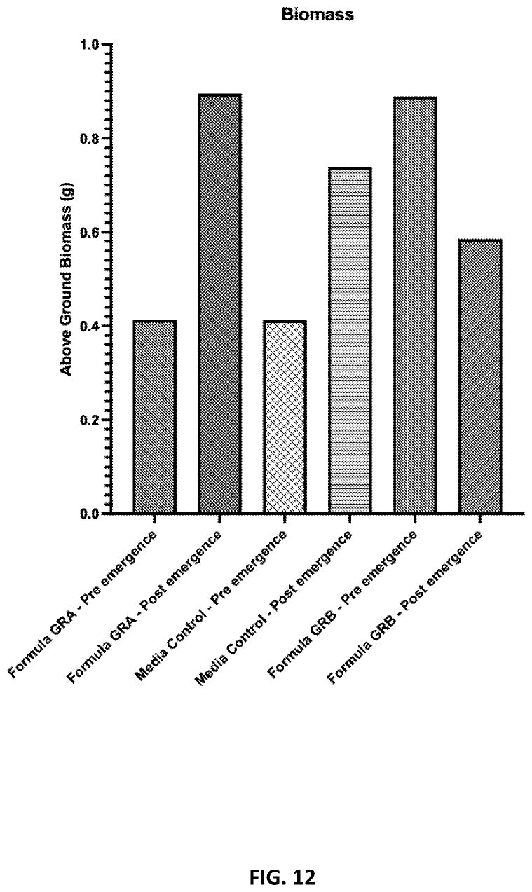

Inoculant formulations, preparation, and application were as described in Example 8. Formula GRA applied post emergence was the most effective biomass enhancers (FIG. 12). Each bar represents the average biomass of the hemp. Seeds were planted and 10 mL of microbial inoculants or controls were applied to the soil either just after planting or after they had emerged from the soil. Biomass was measured by cutting all of the seedlings at their base and weighing them on an analytical scale. Measurements were taken 14 days post germination.

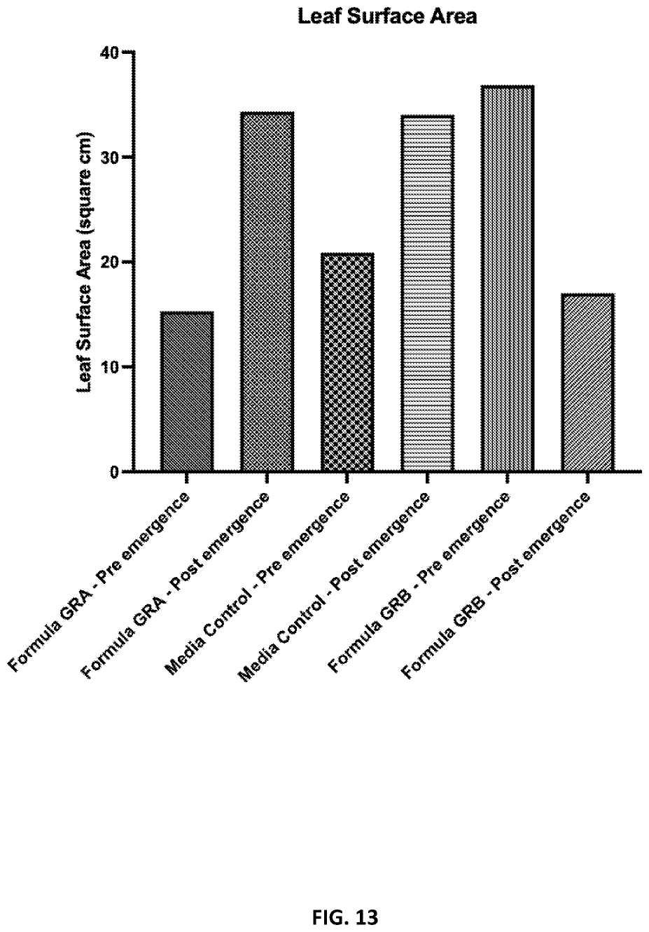

Formula GRB applied pre emergence was the most effective leaf surface area enhancer (FIG. 13). Each bar represents the average leaf surface area of the hemp. Seeds were planted and 10 mL of microbial inoculants or controls were applied to the soil either just after planting or after they had emerged from the soil. Measurements were taken 14 days post germination using the imaging and analytical software ImageJ.

### Example 9: Treatment of Radish Plants with Growth Enhancement Inoculants

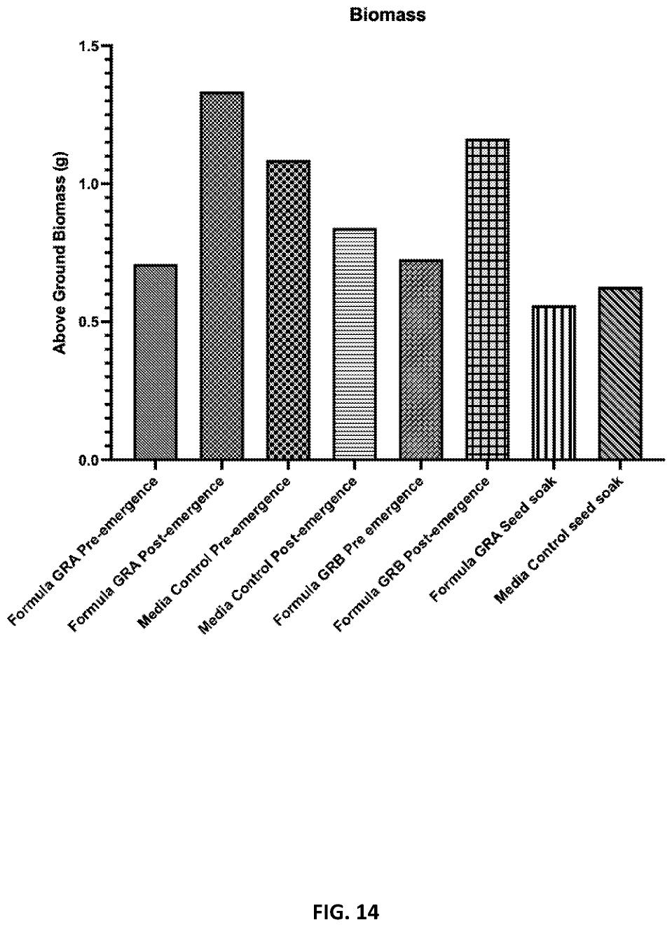

Inoculant formulations, preparation, and application were as described in Example 8. Formula GRA applied post emergence was the most effective biomass enhancer (FIG. 14). Each bar represents the average biomass of the radishes. Seeds were planted and 10 mL of microbial inoculants or controls were applied to the soil either just after planting or after they had emerged from the soil. Seeds were soaked in tubes filled with the appropriate formulation for one hour then planted in the soil. Biomass was measured by cutting all of the seedlings at their base and weighing them on an analytical scale. Measurements were taken 14 days post germination.

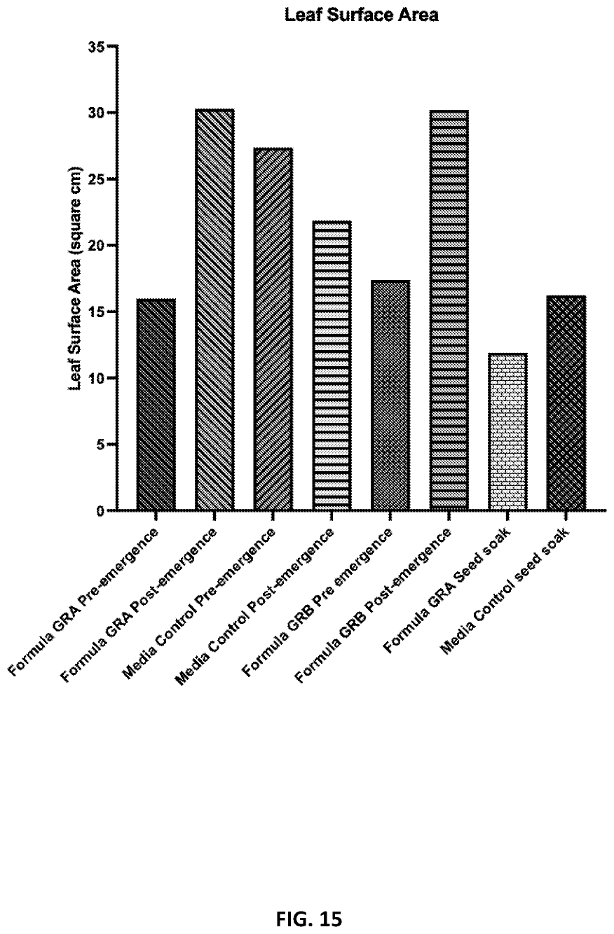

Formula GRA applied post emergence was the most effective leaf surface area enhancer (FIG. 15). Each bar represents the average leaf surface area of the radishes. Seeds were planted and 10 mL of microbial inoculants or controls were applied to the soil either just after planting or after they had emerged from the soil. Seeds were soaked in tubes filled with the appropriate formulation for one hour then planted in the soil. Measurements were taken 14 days post germination.

### Example 9: Treatment of Zinnia Plants with Growth Enhancement Inoculants

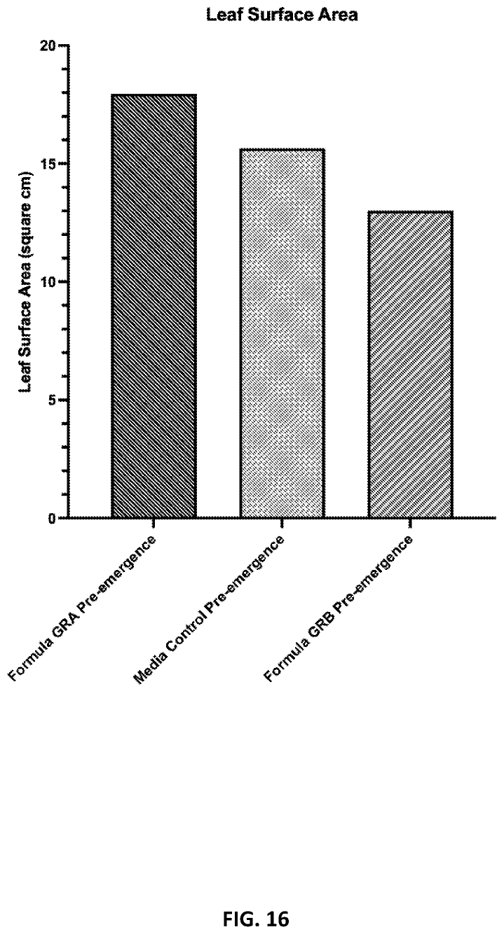

Inoculant formulations, preparation, and application were as described in Example 8. Formula GRA applied pre emergence was the most effective leaf surface area enhancer (FIG. 16). Each bar represents the average leaf surface area of the zinnias. Seeds were planted and immediately treated with a 10 mL soil application. Measurements were taken 14 days post germination. Leaf surface area was measured by using the imaging and analytical software ImageJ.

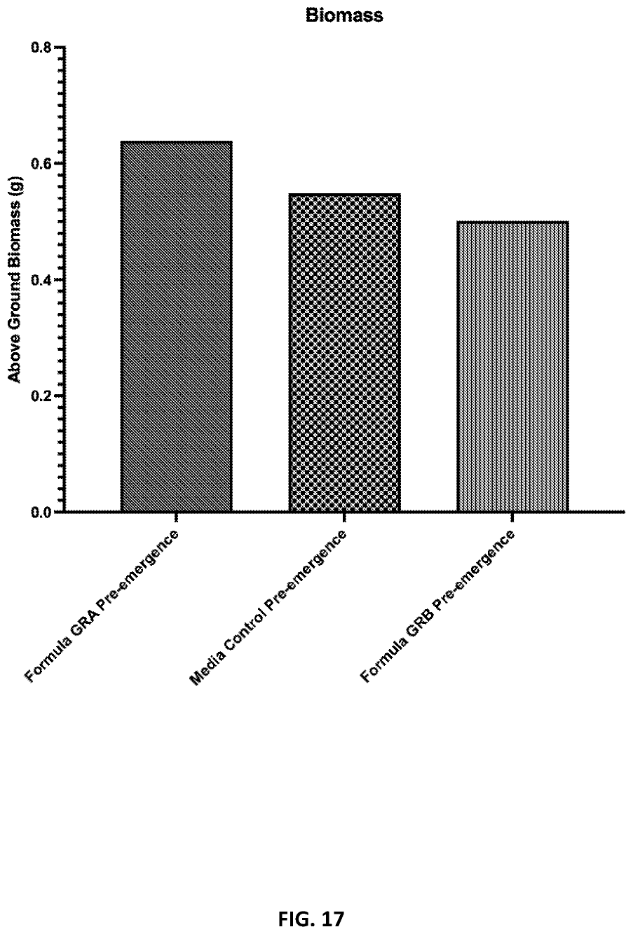

Formula GRA applied pre emergence was the most effective biomass enhancer (FIG. 17). Each bar represents the average biomass of the zinnias. Seeds were planted and treated with a 10 mL soil application immediately. Biomass was measured by cutting all of the seedlings and weighing them on an analytical scale. Measurements were taken 14 days post germination.

### Example 9: Growth Enhancement Inoculants' Effects on Plant Germination Rate

Inoculant formulations and preparation were as described in Example 8.

### 9.1 Corn

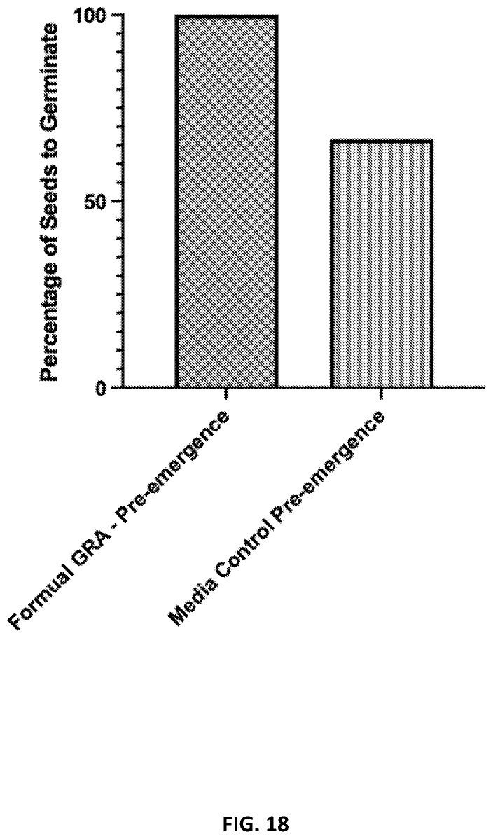

Formula GRA applied pre emergence was a more effective germination enhancer compared to the media control (FIG. 18. Each bar represents the average number of corn seeds that germinated. Seeds were planted and treated with a 10 mL soil application immediately. After 4 days all corn seeds had germinated.

### 9.2 Cosmos

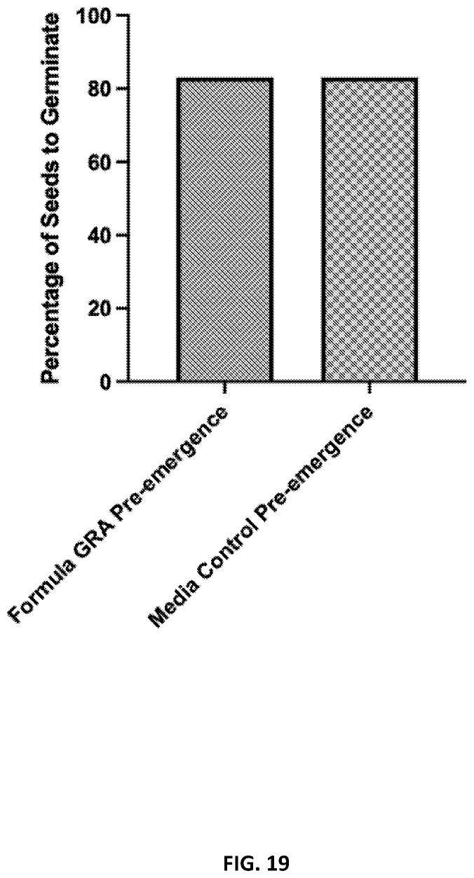

Formula GRA and the Media Control applied pre emergence were equally effective at increasing germination (FIG. 19). Each bar represents the average number of corn seeds that germinated. Seeds were planted and treated with a 10 mL soil application immediately. After 6 days all seeds had germinated.

### 9.3 Legumes

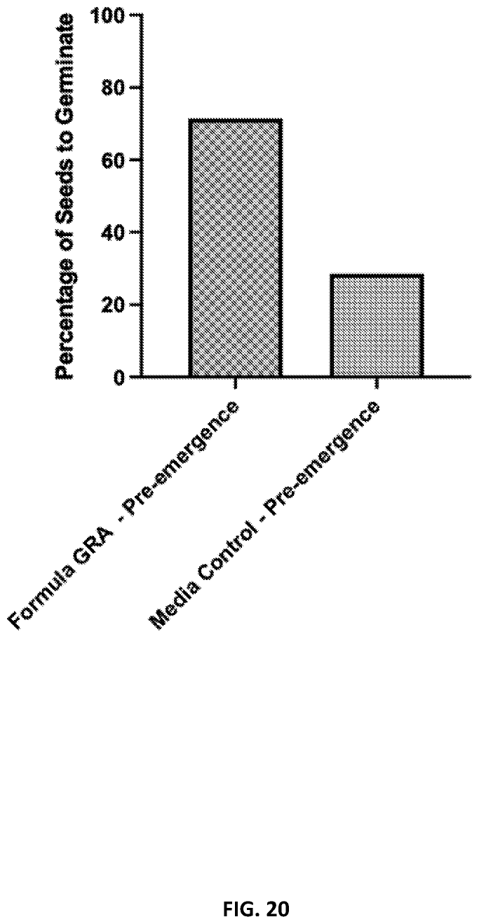

Formula GRA applied pre emergence was more effective at increasing germination compared to the Media Control (FIG. 20). Each bar represents the average number of corn seeds that germinated. Seeds were planted and treated with a 10 mL soil application immediately. After 7 days all seeds had germinated.

### 9.3 Sunflowers

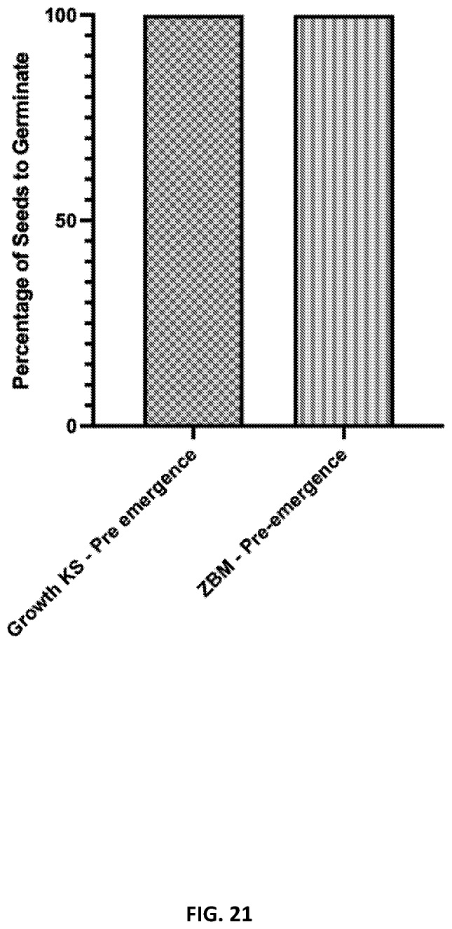

Formula GRA and the Media Control applied pre emergence were equally effective at increasing germination (FIG. 21). Each bar represents the average number of sunflower seeds that germinated. Seeds were planted and treated with a 10 mL soil application immediately. After 6 days all seeds had germinated.

### REFERENCES

1. IBIS. 2019. Fertilizer Manufacturing Industry in the US.
2. Citation Daniel J Sobota et al 2015 Environ. Res. Lett. 10 025006
3. Foucault, A., Vallet, N., Ravalet, N. et al. Occupational pesticide exposure increases risk of acute myeloid leukemia: a meta-analysis of case-control studies including 3,955 cases and 9,948 controls. Sci Rep 11, 2007 (2021). https://doi.org/10.1038/s41598-021-81604-
4. Agricultural chemical exposures and birth defects in the Eastern Cape Province, South Africa A case—control study. Gudrun A Heeren, Joanne Tyler, Andrew Mandeya. Environ Health. 2003; 2: 11. Published online 2003 Oct. 4. doi: 10.1186/1476-069X-2-11
5. Christopher W. Tessum et al., “Inequity in consumption of goods and services adds to racial-ethnic disparities in air pollution exposure,” Proceedings of the National Academy of Sciences (March 2019).
6. C. J. Bronick, R. Lal, Soil structure and management: a review, Geoderma, Volume 124, Issues 1-2, 2005, Pages 3-22, ISSN 0016-7061, https://doi.org/10.1016/j.geoderma.2004.03.005.
7. Herring, Peg. “The Secret Life of Soil.” OSU Extension Service, Oregon State University Extension Service, January 2010, extension.oregonstate.edu/news/secret-life-soil.
8. Ingham, Elaine R. “Soil Bacteria.” Natural Resources Conservation Service Soils, United States Department of Agriculture, www.nrcs.usda.gov/wps/portal/nrcs/detailfull/soils/health/biology/?cid=nrcsl42p2_053862.
9. Ingham, Elaine R. “Soil Fungi.” Natural Resources Conservation Service Soils, United States Department of Agriculture, https://www.nrcs.usda.gov/wps/portal/nrcs/detailfull/soils/health/biology/?cid=nrcsl42p2_05 3864.
10. McNear Jr., D. H. (2013) The Rhizosphere—Roots, Soil and Everything In Between. Nature Education Knowledge 4(3):1
11. J. Handelsman, E. V. Stabb. The Plant Cell October 1996, 8 (10) 1855-1869; DOI: 10.1105/tpc.8.10.1855
12. Hemlata Chauhan, D. J. Bagyaraj, G. Selvakumar, S. P. Sundaram, Novel plant growth promoting rhizobacteria-Prospects and potential, Applied Soil Ecology, Volume 95, 2015, Pages 38-53, ISSN 0929-1393, https://doi.org/10.1016/j.apsoil.2015.05.011.
13. Beneduzi, Anelise et al. “Plant growth-promoting rhizobacteria (PGPR): Their potential as antagonists and biocontrol agents.” Genetics and molecular biology vol. 35, 4 (suppl) (2012): 1044-51. doi:10.1590/s1415-47572012000600020
14. Bonfante, P., Genre, A. Mechanisms underlying beneficial plant-fungus interactions in mycorrhizal symbiosis. Nat Commun 1, 48 (2010). https://doi.org/10.1038/ncomms1046
15. Neher, D. A., & Weicht, T. R. (2018). A plate competition assay as a quick preliminary assessment of disease suppression. Journal of Visualized Experiments, (140).

1. A composition for promoting plant growth or plant resistance to plant pathogens, comprising a mixture of:
at least one first microbial species selected from the group consisting of Azospirillum brasilense, Bacillus amyloliquefaciens, Pseudomonas fluorescens, Rhizophagus irregularis, Bradyrhizobium japonicum, Gluconacetobacter diazotrophicus, Pseudomonas putida, Rhodopseudomonas palustris, Trichoderma hamatum, Trichoderma virens, Bacillus subtilis, Rhizophagus diaphanus, Trichoderma reesei, and Laccaria bicolor; and
at least one second microbial species selected from the group consisting of Azotobacter chroococcum, Herbaspirillum seropedicae, Paenibacillus lentimorbus, Sphingomonas paucimobilis, Pseudomonas chlororaphis, Azorhizobium caulinodans, Bacillus pumilus, Variovorax paradoxus, Rhizophagus intraradices, Rhizophagus clarus, and Trichoderma harzianum.

2. The composition of claim 1, wherein the composition promotes plant growth, and wherein the at least one first microbial species comprise one or more of Azospirillum brasilense, Pseudomonas fluorescens, Rhizophagus irregularis, Rhodopseudomonas palustris, Trichoderma hamatum, and Bacillus subtilis, and the at least one second microbial species comprise one or more of Azotobacter chroococcum, Sphingomonas paucimobilis, Pseudomonas chlororaphis, Variovorax paradoxus, Rhizophagus intraradices, and Bacillus pumilus.

3. The composition of claim 1, wherein the composition promotes plant resistance to plant pathogens, wherein:
a) the at least one first microbial species are P. putida, B. subtilis, P. fluorescens and T. virens, and the at least one second microbial species are B. pumilus and P. chlororaphis;
b) the at least one first microbial species are P. putida, B. subtilis, P. fluorescens, T. virens, and, T. reesei, and the at least one second microbial species are B. pumilus and P. chlororaphis; or
c) the at least one first microbial species are A. brasilense, B. subtilis, and R. palustris, and the at least one second microbial species are A. chroococcum, S. paucimobilis, and V. paradoxus;
d) the at least one first microbial species are A. brasilense, P. fluorescens, B. subtilis, and R. palustris and the at least one second microbial species are A. chroococcum, S. paucimobilis, P. chlororaphis, and V. paradoxus; or
e) the at least one first microbial species are A. brasilense, P. fluorescens, and R. palustris and the at least one second microbial species are A. chroococcum, S. paucimobilis, and P. chlororaphis.

4. The composition of claim 1, wherein the microbial species are lyophilized.

5. A microbial inoculant comprising:
a composition of claim 1; and
water or an aqueous solution.

6. The microbial inoculant of claim 5, further comprising a carbon source.

7. The microbial inoculant of claim 6, wherein the carbon source is a hexose.

8. The microbial inoculant of claim 7, wherein the hexose is glucose.

9. The microbial inoculant of claim 6, wherein the percent weight of the carbon source in the total volume of the microbial inoculant ranges from 9-15%, 10-14%, or 11-13% (w/v).

10. The microbial inoculant of claim 5, further comprising a thickening agent.

11. The microbial inoculant of claim 10, wherein the thickening agent is aloe vera, aloe vera flakes, willow bark extract, silica, pectin, or psyllium husk.

12. The microbial inoculant of claim 10, wherein the percent weight of the thickening agent in the total volume of the microbial inoculant ranges from 2-8%, 3-7%, or 4-6% (w/v).

13. A method for promoting plant growth, comprising contacting
i) an ungerminated seed,
ii) a germinated seed upon planting,
iii) a seedling or roots at transplanting,
iv) a cutting at propagation,
v) a maturing, young plant after planting, or
vi) a maturing plant at a time before or at the time of flowering,
with a microbial inoculant of claim 5, wherein the microbial composition of the microbial inoculate comprises at least one or more of Azospirillum brasilense, Pseudomonas fluorescens, Rhizophagus irregularis, Rhodopseudomonas palustris, Trichoderma hamatum, and Bacillus subtilis, and the at least one second microbial species comprise one or more of Azotobacter chroococcum, Sphingomonas paucimobilis, Pseudomonas chlororaphis, Variovorax paradoxus, Rhizophagus intraradices, and Bacillus pumilus.

14. A method for promoting plant growth, comprising contacting
i) an ungerminated seed,
ii) a germinated seed upon planting,
iii) a seedling or roots at transplanting,
iv) a cutting at propagation,
v) a maturing, young plant after planting, or
vi) a maturing plant at a time before or at the time of flowering,
with a microbial inoculant of claim 5, wherein:
a) the at least one first microbial species are P. putida, B. subtilis, P. fluorescens and T. virens, and the at least one second microbial species are B. pumilus and P. chlororaphis;
b) the at least one first microbial species are P. putida, B. subtilis, P. fluorescens, T. virens, and, T. reesei, and the at least one second microbial species are B. pumilus and P. chlororaphis; or
c) the at least one first microbial species are A. brasilense, B. subtilis, and R. palustris, and the at least one second microbial species are A. chroococcum, S. paucimobilis, and V. paradoxus;
d) the at least one first microbial species are A. brasilense, P. fluorescens, B. subtilis, and R. palustris and the at least one second microbial species are A. chroococcum, S. paucimobilis, P. chlororaphis, and V. paradoxus; or
e) the at least one first microbial species are A. brasilense, P. fluorescens, and R. palustris and the at least one second microbial species are A. chroococcum, S. paucimobilis, and P. chlororaphis.

15. The method of claim 13, wherein the contacting with a mixture of microbial species occurs seasonally, bimonthly, monthly, every 3 weeks, every 2 weeks, weekly, every 5 days, every 3 days, daily, or a similar such period of time appropriate for the grow space and plant species.

16. The method of claim 14, wherein the contacting with a mixture of microbial species occurs seasonally, bimonthly, monthly, every 3 weeks, every 2 weeks, weekly, every 5 days, every 3 days, daily, or a similar such period of time appropriate for the grow space and plant species.
